(19) United States
(12) Patent Application Publication
(10) Pub. No.: US 2011/0160216 A1
(43) Pub. Date: Jun. 30, 2011

(54) Thymidylate Synthase Haplotype is Associated with Tumor Recurrence in Stage II and Stage III Colon Cancer Patients

(75) Inventor: Heinz-Josef Lenz, Los Angeles, CA (US)

(73) Assignee: UNIVERSITY OF SOUTHEM CALIFORNIA

(21) Appl. No.: 12/996,676

(22) PCT Filed: Jun. 10, 2008
(86) PCT No.: PCT/US08/66454

§ 371 Date: Mar. 7, 2011

(51) Int. Cl. C12Q 1/68; A61K 31/513; A61K 31/519; A61P 35/00

(52) U.S. Cl. 514/249; 435/6.11; 514/274

(57) ABSTRACT

The invention provides compositions and methods for determining the likelihood of tumor recurrence following treatment with 5-FU based adjuvant therapy for Stage II or Stage III colon cancer patients. After determining if a patient is less likely to experience tumor recurrence once treated, the invention also provides methods for treating these patients.

### STATEMENT AS TO FEDERALLY SPONSORED RESEARCH

This invention was made with Government support under NIH Grant 5, P30CA14089-271. Accordingly, the Government may have rights in this invention.

### BACKGROUND OF THE INVENTION

Colorectal cancer (CRC) is the third most common malignant tumor in the United States. In the year 2007, an estimated 153,760 new cases will be diagnosed and 52,180 deaths will occur. (Jemal, A. et al. (2007) CA Cancer J Clin 57:43-66.) For patients who undergo successful surgery for colon cancer, additional chemotherapy is recommended in Stage III of the disease. Adjuvant chemotherapy with 5-Fluorouracil (5-FU), Leucovorin and Oxaliplatin (FOLFOX) reduces the rate of recurrence by 41% and the overall death rate by 31% and is the standard of care for Stage III colon cancer patients. (Andre, T. et al. (2004) N EngI J Med 350:2343-51; Kuebler, J. P. et al. (2007) J Clin Oncol 25:2198-204; Moertel, C. G. et al. (1995) Ann Intern Med 122:321-6.) Nevertheless, tumor recurrence after curative resection continues to be a significant problem in the management of patients with colon cancer.

Since its introduction by Heidelberger et al. (Heidelberger, C. et al. (1957) Nature 179:663-6), 5-FU has been the drug of choice for systemic chemotherapy regimens in patients with CRC. Upon entry to the cell, 5-FU is converted to 5-fluoro-2-deoxyuridine monophosphate (FdUMP), and forms a stable ternary complex with TS, the sole de novo source of thymidine in the cell Inhibition of TS rapidly shuts off DNA synthesis and triggers apoptosis and other cell death processes (Danenberg, P. V. et al. (1977) Biochim Biophys Acta 473:73-92; Danenberg, P. V. et al. (2004) Front Biosci 9:2484-94). Methylenetetrahydrofolate reductase (“MTHFR”), another key enzyme of 5-FU metabolism, alternates 5-FU sensitivity indirectly by folate pool variations. MTHFR irreversibly converts 5,10-methylenetetrahydrofolate, a substrate of TS, to 5-methyltetrahydrofolate (Cohen, V. et al. (2003) Clin Cancer Res 9:1611-5; Ulrich, C. M. et al. (2001) Blood 98:231-4), which is essential for the methylation of homocysteine to methionine and is required for the synthesis of S-adenosylmethionine.

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568.

Most studies have consistently agreed that TS expression varies considerably among tumors and that the response rates of various tumors towards 5-FU based chemotherapy regimens are inversely related to intratumoral TS mRNA and protein expression (Pullarkat, S. T. et al. (2001) Pharmacogenomics J 1:65-70; Salonga, D. et al. (2000) Clin Cancer Res 6:1322-7). Although conflicting results have been reported (Edler, D. et al. (2002) J Clin Oncol 20:1721-8; Kornmann, M. et al. (2003) Clin Cancer Res 9:4116-24), higher TS levels in tumors have generally been associated with worse response to 5-FU based chemotherapy regimens (Pullarkat, S. T. et al. (2001) Pharmacogenomics J 1:65-70; Kralovanszky, J. et al. (2002) Oncology 62:167-74; Nakagawa, T. et al. (2002) Lung Cancer 35:165-70).

Three polymorphisms in the TS untranslated regions (UTRs) have been identified. TS promoter enhancer region polymorphism (TS 2R/3R repeat) is a tandem repeat upstream of the TS translational start site containing either double (2R) or triple (3R) repeats of 28 bp sequences. These tandem repeats have been found to be associated with the autoregulation of TS transcription and translation (Pullarkat, S. T. et al. (2001) Pharmacogenomics J 1:65-70; Horie, N. et al. (1995) Cell Struct Funct 20:191-7; Kawakami, K. et al. (1999) Anticancer Res 19:3249-52; Kawakami, K. et al. (2001) Clin Cancer Res 7:4096-101). More recently, additional functional variants within the 5′-UTR region of the TS gene have been identified and TS 2R/3R repeat is now studied together with a G to C single nucleotide polymorphism within the second repeat of the 3R allele (TSER 3R G/C) (Kawakami, K. et al. (2003) Cancer Res 63:6004-7; Mandola, M. V. et al. (2003) Cancer Res 63:2898-904). In fact, TSER 3RC/3RC genotype caused a lower transcriptional activity of TS, comparable to the TS 2R/2R genotype (Kawakami, K. et al. (2003) Cancer Res 63:6004-7). Another functional TS polymorphism is a 6 bp deletion/insertion within the 3′-UTR region of the TS gene (Mandola, M. V. et al. (2004) Pharmacogenetics 14:319-27). TS 1494del6 bp has been shown to decrease RNA stability and therefore influence TS mRNA and TS protein expression in vitro (Mandola, M. V. et al. (2004) Pharmacogenetics 14:319-27).

Although considerable research correlating gene expression and/or polymorphisms has been reported, much work remains to be done. This invention supplements the existing body of knowledge and provides related advantages as well.

### SUMMARY OF THE INVENTION

This invention provides a method for identifying a Stage II or Stage III colon cancer patient that is less likely to experience tumor recurrence following treatment with 5-FU based adjuvant therapy, comprising screening a suitable tissue or cell sample isolated from the patient for the thymidylate synthase (TS) haplotype comprising 5′ UTR TS (high, intermediate or low) and 3′ UTR TS (high or low) polymorphisms, wherein 5′ UTR TS low and 3′ UTR TS low; 5′ UTR TS low and 3′ UTR TS high; or 5′ UTR TS intermediate and 3′ UTR TS low, respectively, identifies the patient as less likely to experience tumor recurrence following 5-FU based adjuvant therapy, as described in Table 1.

Also provided herein is a method for identifying a Stage II or Stage III colon cancer patient that is more likely to experience tumor recurrence following treatment with 5-FU based adjuvant therapy, comprising screening a suitable tissue or cell sample isolated from the patient for the thymidylate synthase (TS) haplotype comprising 5′ UTR TS (high, intermediate or low) and 3′ UTR TS (high or low) polymorphisms, wherein 5′ UTR TS intermediate and 3′ UTR TS high; 5′ UTR TS high and 3′ UTR TS low; or 5′ UTR TS high and 3′ UTR TS high, respectively, identifies the patient as more likely to experience tumor recurrence following 5-FU based adjuvant therapy. See, e.g., Table 1.

Further provided herein is a method for selecting a therapy comprising 5-FU based adjuvant therapy for a Stage II or Stage III colon cancer patient, comprising screening a suitable cell or tissue sample isolated from the patient for the thymidylate synthase (TS) haplotype comprising 5′ UTR TS (high, intermediate or low) and 3′ UTR TS (high or low) polymorphisms, wherein 5′ UTR TS low and 3′ UTR TS low; 5′ UTR TS low and 3′ UTR TS high; or 5′ UTR TS intermediate and 3′ UTR TS low, respectively, selects the patient for said therapy, See, e.g., Table 1.

TABLE 1
Predictive Polymorphisms for Tumor Recurrence Following 5-FU Based Therapy

| | Expression | | |
|---|---|---|---|
| Haplotype | Group | Predictive Genotype | Measured Response |
| 5′ UTR TS low and | Low | 2R/2R, 2R/3R(C) or 3R(C)/3R(C) for 5′ UTR TS | Less Likely to Experience |
| 3′ UTR TS low | | −6 bp/−6 bp or −6 bp/+6 bp for 3′ UTR TS | Tumor Recurrence |
| 5′ UTR TS low and | Low | 2R/2R, 2R/3R(C) or 3R(C)/3R(C) for 5′ UTR TS | Less Likely to Experience |
| 3′ UTR TS high | | +6 bp/+6 bp for 3′UTR TS | Tumor Recurrence |
| 5′ UTR TS intermediate and | Low | 2R/3R(G) or 3R(C)/3R(G) for 5′ UTR TS | Less Likely to Experience |
| 3′ UTR TS low | | −6 bp/−6 bp or −6 bp/+6 bp for 3′ UTR TS | Tumor Recurrence |
| 5′ UTR TS intermediate and | High | 2R/3R(G) or 3R(C)/3R(G) for 5′ UTR TS | More Likely to Experience |
| 3′ UTR TS high | | +6 bp/+ 6 bp for 3′ UTR TS | Tumor Recurrence |
| 5′ UTR TS high and | High | 3R(G)/3R(G) for 5′ UTR TS | More Likely to Experience |
| 3′ UTR TS low | | −6 bp/−6 bp or −6 bp/+6 bp for 3′ UTR TS | Tumor Recurrence |
| 5′ UTR TS high and | High | 3R(G)/3R(G) for 5′ UTR TS | More Likely to Experience |
| 3′ UTR TS high | | +6 bp/+6 bp for 3′UTR TS | Tumor Recurrence |

Yet further provided is a method for treating a colon cancer patient comprising the administration of an effective amount of 5-FU based adjuvant therapy, the method comprising determining the TS haplotype comprising 5′ UTR TS (high, intermediate or low) and 3′ UTR TS (high or low), of the patient from a suitable patient sample, and administering an effective amount of 5-FU based adjuvant therapy to a patient having the TS haplotype of 5′ UTR TS low and 3′ UTR TS low; 5′ UTR TS low and 3′ UTR TS high; or 5′ UTR TS intermediate and 3′ UTR TS low, respectively, thereby treating the patient. See, e.g., Table 1.

### BRIEF DESCRIPTION OF THE DRAWINGS

FIG. 1, panels (a), (b) and (c), show the 5′ UTR TS (TS 2R/3R repeat and TSER 3R G/C) and the 3′ UTR TS 1494del6 bp polymorphisms predict recurrence-free survival in patients with Stage II or Stage III colon cancer. Panel (a) show that 5′ UTR TS polymorphism predicts recurrence-free survival. Panel (b) shows that 5′ UTR and 3′UTR polymorphism combinations predict recurrence-free survival. Panel (c) shows that high expression and low expression groups of the 5′UTR and 3′ UTR TS polymorphisms predict recurrence-free survival. See Tables 1 and 5 for low expression and high expression groups. The designation (n) represents the number of patients. The X-axis indicates the number of years since a patient was diagnosed with Stage II or Stage III colon cancer. The Y-axis indicates the estimated probability of a patient being recurrence free. The adjusted P value for Panels (a), (b) and (c) are 0.013, 0.044 and 0.003, respectively.

### DETAILED DESCRIPTION OF THE EMBODIMENTS

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### DEFINITIONS

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

Fluorouracil (5-FU) belongs to the family of therapy drugs call pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N[4[[(2-amino-5-formyl1,4,5,6,7,8hexahydro4oxo6-pteridinyl)methyl]amino]benzoyl], calcium salt (1:1).

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“FOLFOX” is an abbreviation for a type of combination therapy that is used to treat cancer. This therapy includes 5-FU, oxaliplatin and leucovorin. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on Jan. 16, 2008.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

The term “adjuvant” therapy refers to administration of a therapy or chemotherapeutic regimen to a patient after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

The phrase “first line” or “second line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

In one aspect, the term “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “genetic marker” refers to an allelic variant of a polymorphic region of a gene of interest and/or the expression level of a gene of interest.

As used herein, the term “gene of interest” intends the gene identified as the thymidylate synthase (TS) or the methylenetetrahydrofolate reductase (MTHFR).

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

A “haplotype” is a set of alleles of a group of closesly linked genes which are usually inherited as a unit. The term “allelic variant of a polymorphic region of the gene of interest” refers to a region of the gene of interest having one of a plurality of nucleotide sequences found in that region of the gene in other individuals.

The term “genotype” refers to the specific allelic composition of an entire cell or a certain gene and in some aspects a specific polymorphism associated with that gene, whereas the term “phenotype” refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Expression” as applied to a gene, refers to the differential production of the mRNA transcribed from the gene or the protein product encoded by the gene. A differentially expressed gene may be over expressed (high expression) or under expressed (low expression) as compared to the expression level of a normal or control cell, a given patient population or with an internal control gene (house keeping gene). In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample.

In another aspect, the threshold level of a gene is a level of expression below which it has been found in tumors likely to be responsive, or alternatively, non-responsive to the same treatment for a defined cancer type.

The term “expressed” also refers to nucleotide sequences in a cell or tissue which are expressed where silent in a control cell or not expressed where expressed in a control cell.

In another aspect, “expression” level is determined by measuring the expression level of a gene of interest for a given patient population, determining the median expression level of that gene for the population, and comparing the expression level of the same gene for a single patient to the median expression level for the given patient population. For example, if the expression level of a gene of interest for the single patient is determined to be above the median expression level of the patient population, that patient is determined to have high expression of the gene of interest. Alternatively, if the expression level of a gene of interest for the single patient is determined to be below the median expression level of the patient population, that patient is determined to have low expression of the gene of interest.

A “internal control” or “house keeping” gene refers to any constitutively or globally expressed gene whose presence enables an assessment of the gene of interests expression level. Such an assessment comprises a determination of the overall constitutive level of gene transcription and a control for variation in sampling error. Examples of such genes include, but are not limited to, β-actin, the transferring receptor gene, GAPDH gene or equivalents thereof. In one aspect of the invention, the internal control gene is β-actin.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “mismatches” refers to hybridized nucleic acid duplexes which are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

As used herein, the term “5′ UTR TS low” comprises, or alternatively consists essentially of, or yet further consists of, a genotype selected from 5′ UTR 2R/2R, 2R/3R(C), or 3R(C)/3R(C).

As used herein, the term “5′ UTR TS intermediate” comprises, or alternatively consists essentially of, or yet further consists of a genotype selected from 2R/3R(G) or 3R(C)/3R(G).

As used herein, the term “5′ UTR TS high” comprises, or alternatively consists essentially of, or yet further consists of, a genotype 3R(G)/3R(G).

As used herein, the term “3′ UTR TS low” comprises, or alternatively consists essentially of, or yet further consists of, a genotype selected from −6 bp/−6 bp or −6 bp/+6 bp.

As used herein, the term “3 UTR TS high” comprises, or alternatively consists essentially of, or yet further consists of, the genotype +6 bp/+6 bp.

The term “clinical parameters” refers to a reduction or delay in recurrence of the cancer after the initial therapy, time to tumor recurrence (TTR), time to tumor progression (TTP), decrease in tumor load or size (tumor response or TR), progression free survival (PFS), increase median survival time (OS) or decrease metastases.

A “complete response” (CR) to a therapy defines patients with evaluable but non-measurable disease, whose tumor and all evidence of disease had disappeared.

A “partial response” (PR) to a therapy defines patients with anything less than complete response were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

The term “likely to respond” shall mean that the patient is more likely than not to exhibit at least one of the described clinical parameters or treatment responses, identified above, as compared to similarly situated patients without the polymorphism or the gene expression level. Alternatively, “less likely to respond” indicates the patient is less likely than not to exhibit at least one of the described clinical parameters or treatment responses, identified above, as compared to similarly situated patients without the polymorphism or gene expression level.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

The term “whole blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

### Descriptive Embodiments

This invention provides a method for identifying a Stage II or Stage III colon cancer patient that is less likely to experience tumor recurrence following treatment with 5-FU based adjuvant therapy, comprising, or alternatively consisting essentially of, or yet further consisting of, screening a suitable tissue or cell sample isolated from the patient for the thymidylate synthase (TS) haplotype comprising 5′ UTR TS (high, intermediate or low) and 3′ UTR TS (high or low) polymorphisms, wherein 5′ UTR TS low and 3′ UTR TS low; 5′ UTR TS low and 3′ UTR TS high; or 5′ UTR TS intermediate and 3′ UTR TS low, respectively, identifies the patient as less likely to experience tumor recurrence following 5-FU based adjuvant therapy.

Also provided herein is a method for identifying a Stage II or Stage III colon cancer patient that is more likely to experience tumor recurrence following treatment with 5-FU based adjuvant therapy, comprising, or alternatively consisting essentially of, or yet further consisting of, screening a suitable tissue or cell sample isolated from the patient for the thymidylate synthase (TS) haplotype comprising 5′ UTR TS (high, intermediate or low) and 3′ UTR TS (high or low) polymorphisms, wherein 5′ UTR TS intermediate and 3′ UTR TS high; 5′ UTR TS high and 3′ UTR TS low; or 5′ UTR TS high and 3′ UTR TS high, respectively, identifies the patient as more likely to experience tumor recurrence following 5-FU based adjuvant therapy.

Further provided herein is a method for selecting a therapy comprising, or alternatively consisting essentially of, or yet further consisting of, 5-FU based adjuvant therapy for a Stage II or Stage III colon cancer patient, comprising screening a suitable cell or tissue sample isolated from the patient for thymidylate synthase (TS) haplotype comprising 5′ UTR TS (high, intermediate or low) and 3′ UTR TS (high or low) polymorphisms, wherein 5′ UTR TS low and 3′ UTR TS low; 5′ UTR TS low and 3′ UTR TS high; or 5′ UTR TS intermediate and 3′ UTR TS low, respectively, selects the patient for said therapy.

Yet further provided is a method for treating a colon cancer patient comprising, or alternatively consisting essentially of, or yet further consisting of, the administration of an effective amount of 5-FU based adjuvant therapy, the method comprising determining the TS haplotype comprising 5′ UTR TS (high, intermediate or low) and 3′ UTR TS (high or low), of the patient from a suitable patient sample, and administering an effective amount of 5-FU based adjuvant therapy to a patient having the TS haplotype of 5′ UTR TS low and 3′ UTR TS low; 5′ UTR TS low and 3′ UTR TS high; or 5′ UTR TS intermediate and 3′ UTR TS low, respectively, thereby treating the patient.

As alternate embodiments of each of the above noted inventions, the suitable patient sample comprises, or alternatively consists essentially of, or yet further consists of, tissue or cells selected from non-metastatic tumor tissue, a non-metastatic tumor cell, metastatic tumor tissue, a metastatic tumor cell, peripheral blood lymphocytes or whole blood. In a further aspect, the patient sample comprises peripheral blood lymphocytes. In another aspect the patient sample can be normal tissue isolated adjacent to the tumor. In another aspect the patient sample can be normal tissue isolated distal to the tumor or any other normal tissue or bodily fluid such as whole blood or PBLs.

As alternate embodiments of each of the above noted inventions, the 5-FU based adjuvant therapy comprises, or alternatively consists essentially of, or yet further consists of 5-FU only, FOLFOX (5-FU, leucovorin and oxaliplatin) or FOLFIRI (5-FU, leucovorin and CPT-11).

As alternate embodiments of each of the above noted inventions, 5′ UTR TS low comprises, or alternatively consists essentially of, or yet further consists of, a genotype selected from 5′ UTR 2R/2R, 2R/3R(C), or 3R(C)/3R(C). In a further aspect, the 5′ UTR TS intermediate comprises, or alternatively consists essentially of, or yet further consists of a genotype selected from 2R/3R(G) or 3R(C)/3R(G). In a yet further aspect, the 5′ UTR TS high comprises, or alternatively consists essentially of, or yet further consists of, a genotype 3R(G)/3R(G).

As alternate embodiments of each of the above noted inventions, the 3′ UTR TS low comprises, or alternatively consists essentially of, or yet further consists of, a genotype selected from −6 bp/−6 bp or −6 bp/+6 bp. In another aspect, the 3 UTR TS high comprises, or alternatively consists essentially of, or yet further consists of, the genotype +6 bp/+6 bp.

Any suitable method for identifying the genotype in the patient sample can be used and the inventions described herein are not to be limited to these methods. For the purpose of illustration only, the genotype is determined by a method comprising, or alternatively consisting essentially of, or yet further consisting of, hybridization, PCR or more specifically, PCR-RFLP.

### Diagnostic Methods

The invention further provides diagnostic methods, which are based, at least in part, on determination of the identity of the polymorphic region of the genes identified herein.

### Polymorphic Region

For example, information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support is used as a support capable of binding of a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. No. 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. No. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. Nos. 5,576,180; 6,106,777 or 7,179,600) A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. No. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. No. 6,811,982; 6,835,541 or 7,125,673). DNA is first treated with sodium bisulfite, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. Nos. 5,882,856; 6,531,282 or 7,118,867). By targeting multiple genes at once, additional information may be gained from a single test run that otherwise would require several times the reagents and more time to perform. Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. No. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. Nos. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. No. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₘ of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S, and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genetic polymorphisms identified herein. The prognostic panel comprises probes or primers that can be used to amplify and/or for determining the molecular structure of the polymorphisms identified herein. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the TS gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

The invention further provides methods of treating patients having solid malignant tissue mass or tumor from a gastrointestinal cancer, e.g., rectal cancer, colorectal cancer, colon cancer, gastric cancer, and esophageal cancer. In another aspect, the invention provides methods for treating patients having Stage II colon cancer or Stage III colon cancer. In a further aspect, the above cancers are non-metastatic or metastatic.

In one aspect of the invention, the 5-FU based adjuvant therapy comprises, or alternatively consists essentially of, or yet further consists of administration of a pyrimidine based antimetabolite chemotherapy drug and a platinum based chemotherapy drug, e.g., 5-FU alone or alternatively 5-fluorouracil and oxaliplatin or FOLFOX or equivalents thereof, in an amount effective to treat the cancer and by any suitable means and with any suitable formulation as a composition and therefore includes a carrier such as a pharmaceutically acceptable carrier.

In another aspect, the 5-FU based adjuvant therapy comprises, or alternatively consists essentially of, or yet further consists of administration of a pyrimidine based antimetabolite chemotherapy drug based therapy, including, but not limited to FOLFOX (5-FU, leucovorin and oxaliplatin); FOLFIRI (5-FU, leucovorin and irinotecan) or 5-FU and leucovorin alone in an amount effective to treat the cancer and by any suitable means and with any suitable formulation as a composition and therefore includes a carrier such as a pharmaceutically acceptable carrier.

Accordingly, a formulation comprising the necessary chemotherapy or chemical equivalent thereof is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the invention, the method for treating a patient further comprises, or alternatively consists essentially of, or yet further consists of surgical resection of a metastatic or non-metastatic solid malignant tumor and, in some aspects, in combination with radiation. Methods for treating said tumors derived from a gastrointestinal cancer, e.g., rectal cancer, colorectal cancer, colon cancer, gastric cancer, esophageal cancer, Stage II colon cancer or Stage III colon cancer by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The invention provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Chemotherapeutic formulations of the present invention can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject®NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Jett Corp (Minneapolis, Minn., available at mediject.com).

Various delivery systems are known and can be used to administer a chemotherapeutic agent of the invention, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a medicament comprising an effective amount of a chemotherapeutic as described herein for treatment of a human cancer patient having the polymorphism of the gene of interest as identified in the experimental examples.

### Kits

As set forth herein, the invention provides diagnostic methods for determining the polymorphic region of the gene of interest. In some embodiments, the methods use probes or primers comprising nucleotide sequences which are complementary to the gene of interest. Accordingly, the invention provides kits for performing these methods as well as instructions for carrying out the methods of this invention such as collecting tissue and/or performing the screen, and/or analyzing the results, and/or administration of an effective amount of a 5-FU based adjuvant therapy or equivalents of each thereof. These can be used alone or in combination with other suitable chemotherapy or biological therapy.

In an embodiment, the invention provides a kit for determining whether a subject is likely responsive to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene. Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S, and Thompson, eds., (1991) GENETICS IN MEDICINE, W B Saunders Co., Philadelphia, Pa. This is useful, e.g., in forensic studies.

The invention now being generally described, it will be more readily understood by reference to the following example which is included merely for purposes of illustration of certain aspects and embodiments of the present invention, and are not intended to limit the invention.

### Experimental Details

### Patients

One hundred and ninety-seven (n=197) with locally advanced colon cancer who were treated with 5-FU-based adjuvant chemotherapy [bolus 5-FU: n=30, (15%); infusimal 5-FU: n=162, (85%)] either at the University of Southern California/Norris Comprehensive Cancer Center (USC/NCCC) or at the Los Angeles County/University of Southern California Medical Center (LAC/USCMC), between 1987 and 2007, were eligible for the present study. This study was conducted at the USC/NCCC and approved by the Institutional Review Board of the University of Southern California for Medical Sciences. Patient data were collected retrospectively through chart review. Informed consent was signed by all patients involved in the study.

### Genotyping

Whole blood was collected and genomic DNA was extracted using the QIAamp kit (Qiagen, CA, USA). Samples were tested using polymerase chain reaction restriction fragment length polymorphism (PCR-RFLP) technique. Briefly, forward and reverse primers were used for PCR amplification (see Table 7), PCR products were digested by restriction enzymes (New England Biolab, Massachusetts, USA) and alleles were separated using a 4% NuSieve ethidium bromide stained agarose gel. TS and MTHFR polymorphisms were tested by PCR, followed by allele-specific restriction enzyme digestion method, as previously described (Mandola, M. V. et al. (2003) Cancer Res 63:2898-904; Mandola, M. V. et al. (2004) Pharmacogenetics 14:319-27; Markan, S. et al. (2007) Mol Cell Biochem 302:125-31; Stern, L. L. et al. (2000) Cancer Epidemiol Biomarkers Prev 9:849-53).

### Statistical Analysis

The primary endpoint in this study was time to tumor recurrence (TTR) in Stage II/III colon cancer patients, which was defined as the time from the date of diagnosis of Stage II/III colon cancer to the date of first recurrence, death, or until last contact if the patient was free of any tumor recurrence at the time of last contact. If a patient had not recurred, then TTR was censored at the time of death or at the last follow-up. The associations of time to tumor recurrence with patient's clinico-pathologic characteristics (age, sex, race, Stage, tumor grade, T-stage, N-stage and type of adjuvant chemotherapy) were assessed using univariate survival analyses (log-rank test).

Polymorphisms of TS and MTHFR were coded as categorical variables according to their genotypes. The associations between TS and MTHFR polymorphisms and time to recurrence were examined using Kaplan-Meier curves and log-rank test. The distributions of polymorphisms across demographic characteristics were examined using Fisher's exact test. In the univariate survival analysis, the Pike estimate of relative risk (RR) and its associated 95% confidence interval (95% CI) was based on the log-rank test.

The Cox proportional hazards regression model with stratification factors (race, stage, and type of adjuvant therapy) was fitted to re-evaluate the association between polymorphisms and time to recurrence considering the imbalances in the distributions of baseline characteristics. P values of the log-likelihood ratio test were obtained from the modeling. Interactions between polymorphisms and gender, race, stage, and type of adjuvant therapy on time to recurrence were tested by comparing corresponding likelihood ratio statistics between the baseline and nested Cox proportional hazards models that included the multiplicative product terms (Rothman (1998) Modern epidemiology, Lippincott-Raven). Linkage disequilibrium among polymorphisms in TS and MTHFR genes was assessed using D′ and r² values and haplotype frequencies of two genes were inferred using the program Haploview version 3.32 (see web address: broad.mit.edu/mpg/haploview) and Phase version 2.1 when appropriate (Stephens, M. et al. (2005) Am J Hum Genet 76:449-62; Stephens, M. et al. (2001) Am J Hum Genet 68:978-89).

All statistical tests were two-sided. Analyses were performed using the SAS statistical package version 9.1 (SAS Institute Inc. Cary, N.C., USA).

### Experimental Results

The experimental group of patient consisted of 82 women (42%) and 115 men (58%) with a median age of 59 years (range: 22-87 years). Of the 197 patients, 80 (40.6%) had tumor recurrence, and the probability of a 3-year recurrence was 0.21±0.05 and 0.45±0.05 in patients with stage II and stage III colon cancer, respectively. Of these 80 patients, 37 (46%) had tumor recurrence in the liver; 34 (42%), in the lung; 40 patients (46%) showed peritoneal carcinomatosis; and 25 (31%) had recurrence in other organs. Thirty-three patients showed one site of recurrence (41%); 26 patients (32%) displayed two; and 21 patients (26%), three or more. Of the 197 patients, 43 (29%) died, and the estimate of the median overall survival was not reached.

Eastern Cooperative Oncology Group Performance Status (ECOG PS) was available for 91% (179/197) of all patients in the cohort. A total of 125 patients (70%) had an ECOG PS of 0, and 26 patients (30%) had an ECOG PS of 1.

Patients with Stage II disease were less likely to present recurrence compared to patients who were diagnosed with Stage III (log-rank test, p=0.018). No significant associations between other demographic and clinico-pathologic parameters and TTR was observed (Table 2). Polymorphisms of TS and MTHFR were not associated with demographic (age and gender), clinical (adjuvant chemotherapy), or pathologic characteristics (tumor stage, grade, or lymph node status).

TABLE 2
Demographic and clinicopathologic characteristics and time to tumor recurrence in
patients with locally advanced colon cancer

| | | | | | | Probablility ± | |
|---|---|---|---|---|---|---|---|
| | | Median time to | | | | SE^{a} | |
| | | recurrence (TTR) | | | | of 3-year | |
| | n | years (95% Cl) | | Relative risk (95% Cl) | | recurrence | P value^{c} |
| Age, years | | | | | | | 0.59 |
| ≦50 | 46 | 4.8 | (2.6, 6.8^{b}) | 1 | (Reference) | 0.42 ± 0.08 | |
| >50 | 151 | 6.6 | (4.0, 16.8^{b}) | 0.87 | (0.52, 1.46) | 0.35 ± 0.04 | |
| Sex | | | | | | | 0.69 |
| Male | 115 | 6.6 | (3.4, 12.4^{b}) | 1 | (Reference) | 0.37 ± 0.05 | |
| Female | 82 | 5.9 | (4.8, 16.8^{b}) | 0.91 | (0.58, 1.43) | 0.36 ± 0.06 | |
| Race | | | | | | | 0.70 |
| White | 112 | 5.9 | (4.0, 16.8^{b}) | 1 | (Reference) | 0.34 ± 0.05 | |
| African-American | 12 | 2.6 | (0.8, 7.2^{b}) | 1.54 | (0.66, 3.62) | 0.54 ± 0.16 | |
| Asian | 30 | 7.1 | (1.5, 10.6^{b}) | 1.02 | (0.54, 1.93) | 0.41 ± 0.10 | |
| Hispanic | 43 | 10.4^{b} | (2.7, 10.4^{b}) | 0.87 | (0.47, 1.60) | 0.36 ± 0.09 | |
| T stage | | | | | | | 0.27 |
| T1^{d} | 2 | | | | | | |
| T2^{d} | 15 | 7.4^{b} | (7.4^{b}) | 1 | (Reference) | 0.23 ± 0.12 | |
| T3 | 156 | 6.6 | (3.9, 16.8^{b}) | 2.29 | (0.72, 7.31) | 0.37 ± 0.04 | |
| T4 | 22 | 4.8 | (1.7, 10.7^{b}) | 3.26 | (0.91, 11.69) | 0.46 ± 0.12 | |
| Tx | 4 | 2.7 | (1.3, 11. 3^{b}) | 3.21 | (0.64, 16.06) | 0.50 ± 0.25 | |
| N stage | | | | | | | 0.044 |
| N0 | 72 | 9.4 | (5.9, 16.8^{b}) | 1 | (Reference) | 0.21 ± 0.05 | |
| N1 | 70 | 6.6 | (2.5, 11. 3^{b}) | 1.67 | (0.96, 2.90) | 0.42 ± 0.06 | |
| N2 | 55 | 5.2 | (1.7, 12.4^{b}) | 2.01 | (1.13, 3.56) | 0.48 ± 0.07 | |
| N of resected lymph | | | | | | | 0.46 |
| nodes | | | | | | | |
| <12 | 65 | 5.4 | (2.7, 12.4^{b}) | 1 | (Reference) | 0.39 ± 0.06 | |
| ≧12 | 132 | 7.1 | (4.8, 16.8^{b}) | 0.85 | (0.54, 1.33) | 0.36 ± 0.05 | |
| Adjuvant therapy | | | | | | | 0.75 |
| 5-FU | 133 | 6.6 | (4.8, 16.8^{b}) | 1 | (Reference) | 0.35 ± 0.04 | |
| 5-FU/LV/Oxaliplatin | 40 | 3.4 | (1.8, 4.2^{b}) | 1.21 | (0.64, 2.29) | 0.47 ± 0.12 | |
| 5-FU/LV/CPT-11 | 23 | 7.1^{b} | (1.8, 7.1^{b}) | 1.20 | (0.61, 2.36) | 0.44 ± 0.11 | |
| Tumor site | | | | | | | 0.87 |
| Left | 97 | 6.6 | (3.5, 16.8^{b}) | 1 | (Reference) | 0.35 ± 0.05 | |
| Right^{d} | 92 | 5.9 | (3.4, 13.3^{b}) | 1.04 | (0.66, 1.63) | 0.37 ± 0.05 | |
| Left and Right^{d} | 3 | | | | | | |
| Differentiation | | | | | | | 0.27 |
| Well^{d} | 7 | | | | | | |
| Moderate^{d} | 122 | 9.4 | (5.2, 16.8^{b)} | 1 | (Reference) | 0.34 ± 0.05 | |
| Moderate/poor | 52 | 5.4 | (2.3, 11.1^{b}) | 1.31 | (0.80, 2.15) | 0.41 ± 0.07 | |
| ECOG performance | | | | | | | 0.22 |
| status | | | | | | | |
| 0 | 125 | 13.3^{b} | (5.2, 13.3^{b}) | 1 | (Reference) | 0.32 ± 0.05 | |
| 1 | 54 | 5.7 | (2.5, 9.4) | 1.36 | (0.83, 2.22) | 0.41 ± 0.07 | |
| Stage | | | | | | | 0.018 |
| II | 72 | 9.4 | (5.9, 16.8^{b}) | 1 | (Reference) | 0.21 ± 0.05 | |
| III | 125 | 5.2 | (2.5, 11.1) | 1.81 | (1.10, 2.98) | 0.45 ± 0.05 | |

^{a}Greenwood SE.
^{b}Estimates were not reached.
^{c}Based on log-rank test.
^{d}Grouped together for the estimates of relative risk and probability ± SE of 3-year recurrence.
5-FU, 5-fluorouacil; CI, confidence interval; CPT-11, cisplatin; ECOG, Eastern Cooperative Oncology Group; LV, leucovorin.

### TS 2R/3R Repeat & TSER 3R G/C SNP and TTR

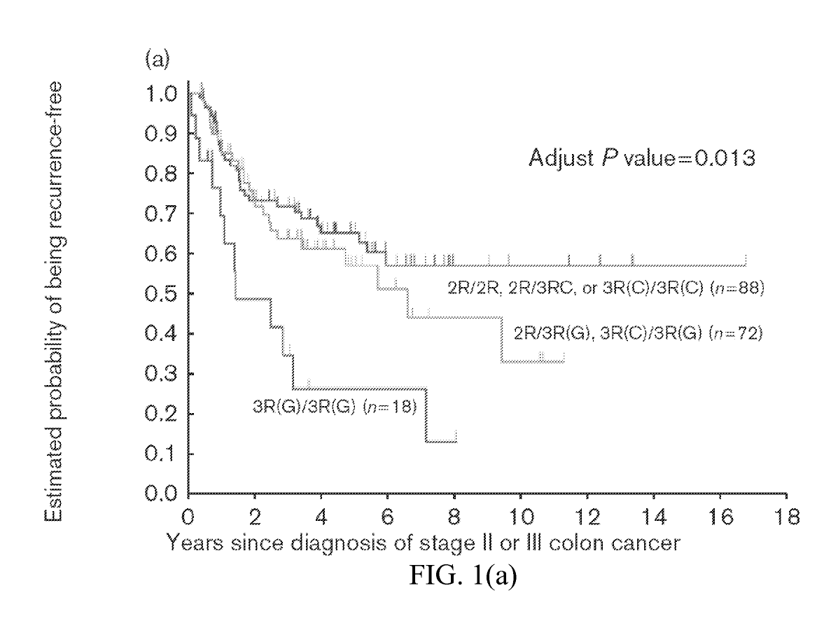

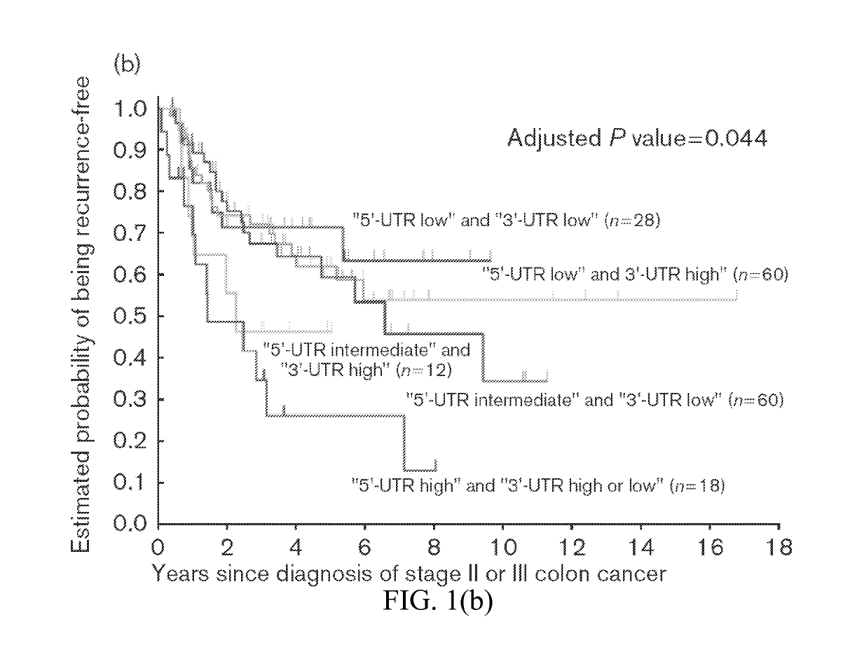

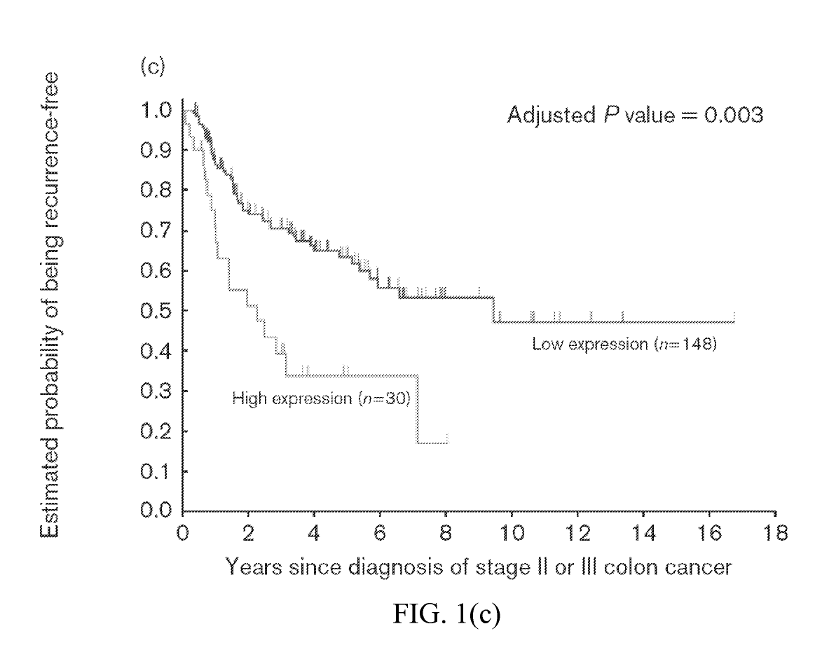

Genotyping for TSER 3R G/C was successful in 178 cases. For 10% of all patients (19/197) genotyping was not successful, because of limited quantity and quality of extracted genomic DNA. The frequency of each genotype was similar to previous reports in white populations, and allelic distribution was in Hardy-Weinberg equilibrium (Table 3). Patients with 2R/2R genotype were at lowest risk to develop tumor recurrence (median TTR: 16.8+ years; 95% CI: 4.0-16.8+), compared to patients carrying the 3R variant (Table 4). However, this association did not reach statistical significance. In joint analysis, TS 2R/3R repeat and TSER 3R G/C SNP showed significant associations with TTR in both univariate (Table 4) and multivariable analysis (Table 5). Patients homozygous for the 3RG allele were at greatest risk to develop tumor recurrence (RR: 3.48 years; 95% CI: 1.61-7.54), compared to patients displaying the 2R/2R, 2R/3RC or 3RC/3RC genotype (adjusted p-value, p=0.013) (Table 5, FIG. 1(a)).

TS 1494de16bp and TTR
Genotyping for TS 1494del6 bp was successful in 179 cases. For 9% of all patients (18/197) genotyping was not successful, because of limited quantity and quality of extracted genomic DNA. The frequency of each genotype was similar to previous reports in white populations, and allelic distribution was in Hardy-Weinberg equilibrium (Table 3). Forty-one percent (73/179) of patients were homozygous for the +6 bp allele, 42% (76/179) were heterozygous for the +6 bp allele, and 17% (30/1 79) showed the −6 bp/−6 bp genotype. TS 1494de16 bp did not show significant associations with TTR (Table 4).

TABLE 3
Allelic distribution of TS and MTHFR polymorphisms by race

| | | | | African- | | | | | |
|---|---|---|---|---|---|---|---|---|---|
| Polymorphism | n | Caucasian | | American | | Asian | | Hispanic | |
| TS-5′UTR | | | | | | | | | |
| 2R/2R | 30 | 24 | (23% | 3 | (27%) | 0 | (0%) | 3 | (8%) |
| 2R/3R | 82 | 46 | (43%) | 5 | (45%) | 12 | (46%) | 19 | (50%) |
| 3R/3R | 69 | 36 | (34%) | 3 | (27%) | 14 | (54%) | 16 | (42%) |
| 3R allele frequency | | 0.557 | | 0.500 | | 0.769 | | 0.671 | |
| P value^{a} | | | | 0.030 | | | | | |
| TS-3′UTR | | | | | | | | | |
| +6 bp/+6 bp | 73 | 46 | (44%) | 4 | (36%) | 3 | (12%) | 20 | (53%) |
| +6 bp/−6 bp | 76 | 45 | (43%) | 5 | (45%) | 12 | (48%) | 14 | (37%) |
| −6 bp/−6 bp | 30 | 14 | (13%) | 2 | (18%) | 10 | (40%) | 4 | (11%) |
| −6 bp allele | | 0.348 | | 0.409 | | 0.640 | | 0.289 | |
| frequency | | | | | | | | | |
| P value^{a} | | | | 0.009 | | | | | |
| MTHFR C677T | | | | | | | | | |
| C/C | 68 | 41 | (38%) | 9 | (82%) | 11 | (44%) | 7 | (18%) |
| C/T | 84 | 57 | (53%) | 2 | (18%) | 8 | (32%) | 17 | (45%) |
| T/T | 29 | 9 | (8%) | 0 | (0%) | 6 | (24%) | 14 | (37%) |
| T allele frequency | | 0.350 | | 0.091 | | 0.400 | | 0.592 | |
| P value^{a} | | | | <0.001 | | | | | |
| MTHFR A1298C | | | | | | | | | |
| A/A | 105 | 50 | (47%) | 6 | (55%) | 15 | (60%) | 34 | (89%) |
| A/C | 65 | 46 | (43%) | 5 | (45%) | 10 | (40%) | 4 | (11%) |
| C/C | 10 | 10 | (9%) | 0 | (0%) | 0 | (0%) | 0 | (0%) |

| | | | | |
|---|---|---|---|---|
| C allele frequency | 0.311 | 0.227 | 0.200 | 0.053 |
| P value^{a} | | <0.001 | | |

^{a}Based Fisher's exact test.
Ts, thymidylate synthase; MTHFR, methylenetetrahydrofolate reductase; UTR, untranslated regions.

TABLE 4
Polymorphisms of genes in folic pathway and time to recurrence
in patients with locally advanced colon cancer

| | All patients | | | | | | |
|---|---|---|---|---|---|---|---|
| | | Median time | | | | Probability ± | |
| | | to recurrence | | | | Se^{a} of | |
| | | (TTR) years | | Relative risk | | 3-year | P |
| | n | (95% CI) | | (95% CI) | | recurrence | value^{c} |
| TS-5′UTR | | | | | | | 0.79 |
| 2R/2R | 30 | 16.8^{b} | (4.0, 16.8^{b}) | 1 | (Reference) | 0.27 ± 0.08 | |
| 2R/3R | 82 | 5.9 | (3.2, 12.4^{b}) | 1.21 | (0.62, 2.35) | 0.37 ± 0.06 | |
| 3R/36 | 69 | 6.6 | (2.8, 13.3^{b}) | 1.26 | (0.64, 2.49) | 0.38 ± 0.07 | |
| TS-5′UTR/GC | | | | | | | 0.019 |
| 2R/2R | 30 | 16.8^{b} | (4.0, 16.8^{b}) | 1 | (Reference) | 0.27 ± 0.08 | |
| 2R/3R(C) | 36 | 12.4^{b} | (3.2, 12.4^{b}) | 1.05 | (0.48, 2.30) | 0.31 ± 0.08 | |
| 3R(C)/3R(C) | 22 | 13.3^{b} | (13.3^{b}) | 0.60 | (0.21, 1.71) | 0.26 ± 0.10 | |
| 2R/3(G) | 43 | 11.3^{b} | (2.3, 11.3^{b}) | 1.20 | (0.57, 2.54) | 0.41 ± 0.08 | |
| 3R(G)/3R(C) | 29 | 5.7 | (3.5, 9.4) | 1.11 | (0.48, 2.58) | 0.29 ± 0.10 | |
| 3R(G)/3R(G) | 18 | 1.4 | (1.0, 3.2) | 2.88 | (1.29, 6.45) | 0.65 ± 0.12 | |
| TS-3′UTR | | | | | | | 0.62 |
| +6bp/+6bP | 73 | 16.8^{b} | (3.9, 16.8^{b}) | 1 | (Reference) | 0.32 ± 0.06 | |
| +6b/−6bp | 76 | 6.6 | (4.8, 11.3^{b}) | 1.06 | (0.63, 1.80) | 0.32 ± 0.06 | |
| −6bp/−6bp | 30 | 7.1 | (1.6, 7.7^{b}) | 1.38 | (0.71, 2.67) | 0.48 ± 0.10 | |
| MTHFR C + 677T | | | | | | | 0.11 |
| C/C | 68 | 5.9 | (3.5, 13.3^{b}) | 1 | (Reference) | 0.33 ± 0.06 | |
| C/T | 84 | 5.4 | (2.7, 16.8^{b}) | 1.11 | (0.68, 1.80) | 0.41 ± 0.06 | |
| T/T | 29 | 7.4^{b} | (7.4^{b}) | 0.39 | (0.14, 1.10) | 0.16 ± 0.08 | |
| MTHFR | | | | | | | 0.34 |
| A + 1298C | | | | | | | |
| A/A | 105 | 16.8^{b} | (5.4, 16.8^{b}) | 1 | (Reference) | 0.31 ± 0.05 | |
| A/C | 65 | 5.9 | (2.8, 13.3^{b}) | 1.31 | (0.80, 2.15) | 0.39 ± 0.06 | |
| C/C | 10 | 3.4 | (1.0, 9.0^{b}) | 1.71 | (0.72, 4.09) | 0.40 ± 0.15 | |
| | Patients receiving 5-FU only | | | | | | |
| | | Median time | | | | Probability ± | |
| | | to recurrence | | | | SE^{a} of | |
| | | (TTR) years | | Relative risk | | 3-year | P |
| | n | (95% CI) | | (95% CI) | | recurrence | value^{c} |
| TS-5′UTR | | | | | | | 0.21 |
| 2R/2R | 25 | 16.8^{b} | (5.2, 16.8^{b}) | 1 | (Reference) | 0.20 ± 0.08 | |
| 2R/3R | 57 | 4.8 | (2.0, 12.4^{b}) | 1.94 | (0.88, 4.27) | 0.42 ± 0.07 | |
| 3R/36 | 38 | 6.6 | (3.5, 13.3^{b}) | 1.43 | (0.61, 3.38) | 0.29 ± 0.08 | |
| TS-5′UTR/GC | | | | | | | 0.012 |
| 2R/2R | 25 | 16.8^{b} | (5.2, 16.8^{b}) | 1 | (Reference) | 0.20 ± 0.08 | |
| 2R/3R(C) | 28 | 5.9 | (1.6, 12.4) | 1.79 | (0.74, 4.30) | 0.38 ± 0.10 | |
| 3R(C)/3R(C) | 12 | 13.3^{b} | (13.3^{b}) | 0.46 | (0.10, 2.14) | 0.17 ± 0.11 | |
| 2R/3(G) | 26 | 4.8 | (1.5, 11.3^{b}) | 1.81 | (0.74, 4.42) | 0.44 ± 0.10 | |
| 3R(G)/3R(C) | 17 | 5.7 | (3.5, 9.4) | 1.44 | (0.52, 3.96) | 0.24 ± 0.12 | |
| 3R(G)/3R(G) | 9 | 1.4 | (0.7, 3.6^{b}) | 4.74 | (1.60, 14.05) | 0.61 ± 0.17 | |
| TS-3′UTR | | | | | | | 0.62 |
| +6bp/+6bP | 53 | 16.8^{b} | (5.2, 16.8^{b}) | 1 | (Reference) | 0.28 ± 0.06 | |
| +6b/−6bp | 47 | 5.7 | (3.2, 11.3^{b}) | 1.33 | (0.72, 2.45) | 0.34 ± 0.07 | |
| −6bp/−6bp | 18 | 7.7^{b} | (1.3, 7.7^{b}) | 1.36 | (0.57, 3.25) | 0.43 ± 0.12 | |
| MTHFR C + 677T | | | | | | | 0.23 |
| C/C | 44 | 6.6 | (4.0, 13.3^{b}) | 1 | (Reference) | 0.26 ± 0.07 | |
| C/T | 56 | 5.4 | (2.4, 16.8^{b}) | 1.30 | (0.72, 2.34) | 0.41 ± 0.07 | |
| T/T | 20 | 7.4^{b} | (7.4^{b}) | 0.55 | (0.19, 1.61) | 0.21 ± 0.09 | |
| MTHFR | | | | | | | 0.56 |
| A + 1298C | | | | | | | |
| A/A | 73 | 16.8^{b} | (5.2, 16.8^{b}) | 1 | (Reference) | 0.31 ± 0.06 | |
| A/C | 41 | 5.9 | (3.2, 13.3b) | 1.33 | (0.74, 2.36) | 0.35 ± 0.08 | |
| C/C | 5 | 6.6 | (0.7, 9.0^{b}) | 1.50 | (0.45, 4.98) | 0.40 ± 0.22 | |

^{a}Greenwood SE.
^{b}Estimates were not reached.
^{c}Based on log-rank test.
5-FU, 5-fluorouracil;
CI, confidence interval;
TS, thymidylate synthase;
MTHFR, methylenetetrahydrofolate reductase;
UTR, untranslated regions.

TABLE 5
Multivariable analysis of TS polymorphisms and time to recurrence
in patients with Stage II and Stage III colon cancer

| | | Adjusted RR | | Adjusted |
|---|---|---|---|---|
| | n | (95% CI)^{a} | | P value |
| TS-5′UTR (TS, 2R/3R repeat and TSER | | | | |
| 3R G/C) | | | | |
| 2R/2R, 2R/3R(C), 3R(C)/3R(C) | 88 | 1 | (Reference) | 0.013 |
| (5′-UTR low) | | | | |
| 2R/3R(G), 3R(C)/3R(G) | 72 | 1.32 | (0.75, 2.30) | |
| (5′-UTR intermediate) | | | | |
| 3R(G)/3R(G) (5′-UTR high) | 18 | 3.48 | (1.61, 7.54) | |
| Additive model | | | | |
| 3R(G) | | 1.70 | (1.16, 2.47) | 0.006 |
| TS-3′UTR (TS 1494del6 bp) | | | | |
| −6 bp/−6 bp, −6 bp/+6 bp) | 106 | 1 | (Reference) | 0.40 |
| (3′-UTR low) | | | | |
| +6 bp/+6 bp (3′-UTR high) | 73 | 0.80 | (0.47, 1.36) | |
| TS-5′-UTR low and TS-3′-UTR combined | | | | |
| 5′-UTR low and 3′-UTR low | 28 | 1 | (Reference) | 0.044 |
| 5′-UTR low and 3′-UTR high | 60 | 0.96 | (0.42, 2.20) | |
| 5′-UTR intermediate and 3′-UTR low | 60 | 1.17 | (0.51, 2.68) | |
| 5′-UTR intermediate and 3′-UTR high^{c} | 12 | 2.17 | (0.63, 7.47) | |
| 5′-UTR high and 3′-UTR low^{c} | 17^{b} | 3.41 | (1.33, 8.75) | |
| 5′-UTR high and 3′-UTR high^{c} | 1 | | | |
| Functional status | | | | |
| Low expression | 148 | 1 | (Reference) | 0.003 |
| High expression^{c} | 30 | 2.81 | (1.49-5.30) | |

^{a}Based on Cox proportional hazards model, stratified by stage, race, and type of adjuvant therapy.
^{b}Grouped together in Cox model.
^{c}High-expression group.
CI, confidence interval; RR, relative risk; TS, thymidylate synthase; TSER, TS enhancer region; UTR, untranslated regions.

Multivariable and Combined Analysis of TS 2R/3R Repeat, TSER 3R G/C & TS 1494de16 bp

TS 2R/3R repeat, TSER 3R G/C and TS 1494del6 bp were analyzed jointly, stratified by race, stage of disease and type of adjuvant therapy. Combining the three TS polymorphisms according to their functional significance, the patients could be divided into “5′-UTR low” (2R/2R or 2R/3RC or 3RC/3RC), “5′-UTR intermediate” (2R/3RG or 3RC/3RG), “5′-UTR high” (3RG/3RG), “3′-UTR low” (−6 bp/−6 bp or −6 bp/+6 bp) and “3′-UTR high” (+6 bp/+6 bp). In combination analysis of 5′-UTR and 3′-UTR, patients could be separated into six different “expression” groups (Table 5, FIG. 1(b)). There was a statistically significant relationship between the three polymorphisms and TTR and Kaplan-Meier survival curves showed a significant difference between genotype combinations (adjusted p-value=0.044). Patients with “5′-UTR high” and “3′-UTR low” or “3′-UTR high” were at greatest risk to develop tumor recurrence (RR: 341 years; 95% CI: 1.33-8.75), compared to patients displaying other genotype combinations (adjusted p-value=0.044, log-rank test) (Table 5, FIG. 1(b)). Grouping alleles according to their functional significance, “high expression” variants of all 3 TS polymorphisms were associated with a higher likelihood of developing tumor recurrence compared to ‘low expression’ variants (RR: 2.81 years; 95% CI: 1.49-5.30) (adjusted p-value=0.003) (Table 5, FIG. 1(c)).

### Haplotype Analysis

As previously described, TS 5′-UTR and 3′-UTR polymorphisms are in linkage disequilibrium in the study population (D′=0.511, r²=0.102; 95% CI: 0.33-0.65). Patients harboring the 3RG/+6 bp haplotype were at greatest risk to develop tumor recurrence (RR: 2.25 years; 95% CI: 1.04-4.85), compared to patients with the most prevalent haplotype 2R/+6 bp (reference) (adjusted p-value=0.032) (Table 6).

TABLE 6
Haplotype analysis for TS 5′ UTR and 3′ UTR polymorphisms and
time to recurrence in patients with Stage II/III colon cancer

| | Estimated | | | |
|---|---|---|---|---|
| | haplotype | Actuated RR | | Adjusted |
| TS-5′UTR/TS-3′UTR | frequency (%) | (95% CI)^{a} | | P value |
| 2R/+6 bp | 32.5 | 1 | (Reference) | 0.032 |
| 2R/−6 bp | 7.0 | 0.63 | (0.26, 1.53) | |
| 3RC/+6 bp | 22.3 | 0.68 | (0.39, 1.16) | |
| 3RG/−6 bp | 22.7 | 1.39 | (0.85, 2.28) | |
| 3RC/−6 bp | 8.0 | 1.46 | (0.76, 2.81) | |
| 3RG/+6 bp | 7.4 | 2.25 | (1.04, 4.85) | |

^{a}Based on Cox proportional hazards model, stratified by stage, race, and type of adjuvant therapy.
CI, confidence interval; RR, relative risk; TS, thymidylate synthase; UTR, untranslated regions.

TABLE 7
Primer Sequences, Annealing Temperatures and Restriction
Enzymes for Determining Polymorphisms

| | | | | Temp. |
|---|---|---|---|---|
| Polymorphism | Forward Primer | Reverse Primer | Enzyme | (° C.) |
| TSER 5′-UTR repeat | GTGGCTCCTGCGTTTCCCCC | GCTCCGAGCCGGCCACAGGC | — | 67° |
| | | ATGGCGCGG | | |
| TSER 3R G/C | GTGGCTCCTGCGTTTCCCCC | GCTCCGAGCCGGCCACAGGC | Hae III | 67° |
| | | ATGGCGCGG | | |
| TS1494del6 bp | ATCTGAGGGAGCTGAGT | CAGATAAGTGGCAGTACAGA | Dra I | 58° |

Analysis of Allelic Frequencies and Interactions Between TS 2R/3R Repeat, TSER 3R G/G and TS 1494de16 bp and Sex, Race, Stage, and Type of Adjuvant Therapy on TTR

Allelic distribution of TS and MTHFR polymorphisms were significantly different among the ethnic groups (Table 3). However, no significant differences in the associations between TS by functional status and TTR differed by sex, stage, race and type of adjuvant chemotherapy were observed.

### MTHFR C+677T and A+1298C and TTR

No statistically significant association between MTHFR C+677T and MTHFR A+1298C and TTR (Table 4) was observed. The MTHFR C+677T and MTHFR A+1298C polymorphisms were in strong linkage disequilibrium with D′ value of 1.0 (95% CI: 0.86-1.0) with moderate r² value of 0.194. The three common haplotypes were found and account for 100% of all haplotypes. No significant associations between MTHFR haplotypes and time to recurrence was observed.

### Experimental Discussion

Most studies have consistently agreed that TS expression varies considerably among tumors and that the response rates towards 5-FU based chemotherapy regimens are related to intratumoral TS mRNA and protein expression. Although results have not been consistent (Edler, D. et al. (2002) J Clin Oncol 20:1721-8), higher TS protein and mRNA expression levels in tumors have generally been associated with worse response to 5-FU based chemotherapy regimens (Pullarkat, S. T. et al. (2001) Pharmacogenomics J 1:65-70; Kralovanszky, J. et al. (2002) Oncology 62:167-74; Nakagawa, T. et al. (2002) Lung Cancer 35:165-70; Iacopetta, B. et al. (2001) Br J Cancer 85:827-30; Popat, S. et al. (2004) J Clin Oncol 22:529-36). Recent investigations revealed that TS polymorphisms may be used as a surrogate for intratumoral TS levels in patients treated with 5-FU based chemotherapy (Pullarkat, S. T. et al. (2001) Pharmacogenomics J 1:65-70). Iacopetta et al. investigated the predictive value of TS in 117 patients treated with 5-FU based adjuvant chemotherapy and 104 untreated patients (Iacopetta, B. et al. (2001) Br J Cancer 85:827-30). The authors found that Stage III colon cancer patients with the TS 3R/3R genotype derive less survival benefit from chemotherapy than those with the 2R/2R or 2R/3R genotype (Iacopetta, B. et al. (2001) Br J Cancer 85:827-30).

A comprehensive analysis of TS gene polymorphisms in 197 consecutive patients homogenously treated with 5-FU based adjuvant chemotherapy were analyzed. This study shows that patients can be separated into prognostic groups according to their TS “expression status” as determined indirectly by the comprehensive assessment of 3 functional TS polymorphisms. Furthermore “high-expression” variants of TSER 3R G/C alone or in combination with the TS 1494del6 bp polymorphism, independently predict tumor recurrence in patients with locally advanced colon cancer treated with adjuvant chemotherapy.

In the present study, patients harboring TS 3RG/+6 bp haplotype were at highest risk to develop tumor recurrence, which is consistent with a recent study by Kawakami et al. in adjuvant gastric carcinoma (Kawakami, K. et al. (2005) Clin Cancer Res 11:3778-83). As proposed by Kawakami et al. (2005), “high expression” variants of 5′-UTR and/or 3′-UTR polymorphisms were associated with decreased OS and higher likelihood of tumor relapse. The presence of at least one high TS expression genotype showed independent adverse prognostic role in univariate and multivariate analysis (Kawakami, K. et al. (2005) Clin Cancer Res 11:3778-83), (Hitre, E. et al. (2005) Pharmacogenet Genomics 15:723-30.), identified the TS 2R/3R repeat and the TS1494del6 bp polymorphisms to be prognostic markers in patients with CRC treated with adjuvant chemotherapy. In their study, the G to C SNP within the 5′-UTR and TS haplotype analysis was not performed. Therefore the data presented herein for Stage II and III colon cancer patients confirms and expands their findings for patients with either colon or rectal carcinoma. Contrary to the majority of reports showing an adverse prognostic role of the TS 3R/3R genotype (Pullarkat, S. T. (2001) Pharmacogenomics J 1:65-70; Kawakami, K. et al, (2001) Clin Cancer Res 7:4096-101; Iacopetta, B. et al. (2001) Br J Cancer 85:827-30; Kawakami, K. et al. (2005) Clin Cancer Res 11:3778-83; Villafranca, E. et al. (2001) J Clin Oncol 19:1779-86), a recent study by Jakobsen et al. (Jakobsen, A. (2005) J Clin Oncol; 23:1365-9) demonstrated that 3R/3R genotypes were associated with better response rates to 5-FU based chemotherapy, compared to patients carrying 2R alleles. It has been suggested that a G to C SNP within the 5′-UTR 28 bp region and a 6 bp deletion with the 3′-UTR of the TS gene could account for some of the aforementioned discrepant study results (Mandola, M. V. et al. (2003) Cancer Res 63:2898-904; Mandola, M. V. et al. (2004) Pharmacogenetics 14:319-27; Marcuello, E. et al. (2004) Int J Cancer 112:733-7). In fact, the study reported herein shows that patients harboring the TSER 3RG allele alone or in combination with either 3′-UTR 6 bp-deletion (−6 bp) or 6 bp-insertion (+6 bp) were a greatest risk to develop tumor recurrence, suggesting a predominant role of TSER 3R G/C, compared to 3′-UTR TS 1491del6 bp. This is in contrast to recent observations by Dotor et al. who genotyped 129 patients with colorectal carcinoma (Dotor, E. et al. (2006) J Clin Oncol 24:1603-11). In their study, TS 1494del6 bp has been shown to be a prognostic factor for patients receiving adjuvant 5-FU based chemotherapy and 3R/−6 bp haplotype was found to be associated with more favorable outcome and response. However, it should be noted that their findings were based on a heterogeneous and relatively small study population. In fact, 79.1% (102/129) of all patients genotyped had tumors in the left colon and rectum and 65.1% (84/129) had either Stage III or Stage IV disease. In this context, it should be noted that results of palliative chemotherapy cannot be extrapolated to the adjuvant setting and vice versa. Furthermore it is being increasingly recognized that colon and rectal cancers are separate and distinct disease groups, in terms of clinical outcome and treatment, and therefore need to be considered individually (Konishi, K. et al. (1999) Gut 45:818-21).

The present study also found that polymorphisms in the MTHFR gene tended to be associated with time to tumor recurrence (TTR), although this trend did not reach statistical significance at the 0.05 level. Consistent with previously published reports on the functional significance of MTHFR (Marcuello, E. et al. (2006) Cancer Chemother Pharmacol 57:835-40), patients carrying the MTHFR +677 TIT variant were less likely to show recurrent disease (median TTR: 7.4+ years; 95% CI: 7.4+), compared to patients carrying the C-allele (log-rank test, p=0.11) (Table 3).

As with all retrospective studies, this analysis has potential limitations. First, these findings are based on a study population uniformly treated with 5-FU based chemotherapy. Since all patients included in this study were Stage III or high-risk Stage II disease, it was not possible to assess genotype combinations associated with clinical outcome in an untreated control group; secondly, TS mRNA gene expression or immunohistochemistry (IHC) from microdissected tumor tissue were not performed. Therefore, this analysis is not free of potential biases, and does not account for the loss of heterozygosity (LOH) in the tumor (Zinzindohoue, F. et al. (2001) J Clin Oncol 19:3442; Uchida, K. et al. (2004) Clin Cancer Res 10:433-9). However, it should be noted that IHC is a semiquantitative and subjective method and is limited by the sensitivity of the monoclonal antibody and the tissue handling. Also, measurements of TS mRNA expression do not account for differences in the quality of TS mRNA, which may be altered by translational efficiency and RNA-stability (Kawakami, K. et al. (2003) Cancer Res 63:6004-7; Mandola, M. V. et al. (2004) Pharmacogenetics 14:319-27). In fact, Kawakami et al. demonstrated, that TSER may induce high TS protein expression in the absence of increased TS mRNA levels (Kawakami, K. et al. (2001) Clin Cancer Res 7:4096-101). TS genotyping might overcome those limitations and additionally it would be of major advantage to analyze germline polymorphisms of the host, which offers a quick and non-invasive approach.

Notwithstanding the aforementioned limitations, this study shows that all 3 TS polymorphisms analyzed are in Hardy-Weinberg equilibrium and in linkage equilibrium, as reported previously (Dotor, E. (2006) J Clin Oncol 24:1603-11; Chen, J. et al. (2003) Cancer Epidemiol Biomarkers Prey 12:958-62; Lecomte, T. et al. (2004) Clin Cancer Res 10:5880-8; Ulrich, C. M. et al. (2002) Cancer Res 62:3361-4). Patients harboring the 3RG/+6 bp haplotype were at greatest risk to develop tumor recurrence. Also, “high-expression variants of TSER 3R G/C (TSER 3RG/3RG or 3RG/3RC) alone or in combination with TS 1494del6 bp showed to be adverse prognostic markers in both univariate and multivariable analysis. Findings such as these will not only assist clinicians identify patients who are at high risk, but they will also be critical in selecting more efficient treatment strategies.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying a Stage II or Stage III colon cancer patient that is less likely or more likely to experience tumor recurrence following treatment with 5-FU based adjuvant therapy, comprising screening a suitable tissue or cell sample isolated from the patient for the thymidylate synthase (TS) haplotype comprising 5′ UTR TS (high, intermediate or low) and 3′ UTR TS (high or low) polymorphisms, wherein
a. 5′ UTR TS low and 3′ UTR TS low;
b. 5′ UTR TS low and 3′ UTR TS high; or
c. 5′ UTR TS intermediate and 3′ UTR TS low,
identifies the patient as less likely to experience tumor recurrence following 5-FU based adjuvant therapy or
d. 5′ UTR TS intermediate and 3′ UTR TS high;
e. 5′ UTR TS high and 3′ UTR TS low; or
f. 5′ UTR TS high and 3′ UTR TS high,
identifies the patient as more likely to experience tumor recurrence following 5-FU based adjuvant therapy.

2. (canceled)

3. A method for selecting a therapy comprising 5-FU based adjuvant therapy for a Stage II or Stage III colon cancer patient, comprising screening a suitable cell or tissue sample isolated from the patient for thymidylate synthase (TS) haplotype comprising 5′ UTR TS (high, intermediate or low) and 3′ UTR TS (high or low) polymorphisms, wherein
a. 5′ UTR TS low and 3′ UTR TS low;
b. 5′ UTR TS low and 3′ UTR TS high; or
c. 5′ UTR TS intermediate and 3′ UTR TS low,
selects the patient for said therapy.

4. A method for treating a colon cancer patient comprising the administration of an effective amount of 5-FU based adjuvant therapy, the method comprising:
a. determining the TS haplotype comprising 5′ UTR TS (high, intermediate or low) and 3′ UTR TS (high or low), of the patient from a suitable patient sample, and
b. administering an effective amount of 5-FU based adjuvant therapy to a patient having the TS haplotype of
i. 5′ UTR TS low and 3′ UTR TS low;
ii. 5′ UTR TS low and 3′ UTR TS high; or
iii. 5′ UTR TS intermediate and 3′ UTR TS low,
thereby treating the patient.

5. The method of any of claims 1, 3 or 4, wherein the patient sample comprises tissue or cells selected from non-metastatic tumor tissue, a non-metastatic tumor cell, metastatic tumor tissue, a metastatic tumor cell, peripheral blood lymphocytes or whole blood.

6. The method of any of claims 1, 3 or 4, wherein the patient sample comprises a non-metastatic tumor cell or tissue.

7. The method of any of claims 1, 3 or 4, wherein the patient sample comprises peripheral blood lymphocytes or whole blood.

8. The method of any of claims 1, 3 or 4, wherein the 5-FU based adjuvant therapy comprises 5-FU only, FOLFOX (5-FU, leucovorin and oxaliplatin) or FOLFIRI (5-FU, leucovorin and CPT-11).

9. The method of claim 1, wherein 5′ UTR TS low comprises a genotype selected from 5′ UTR 2R/2R, 2R/3R(C), or 3R(C)/3R(C).

10. The method of claim 1, wherein 5′ UTR TS intermediate comprises a genotype selected from 2R/3R(G) or 3R(C)/3R(G).

11. The method of claim 1 wherein 5′ UTR TS high comprises a genotype 3R(G)/3R(G).

12. The method of claim 1, wherein 3′ UTR TS low comprises a genotype selected from −6 bp/−6 bp or −6 bp/+6 bp.

13. The method of claim 1, wherein 3′ UTR TS high comprises a genotype +6 bp/+6 bp.

14. The method of claim 9, wherein the genotype is determined by a method comprising hybridization or PCR.

15. The method of claim 9, wherein the genotype is determined by a method comprising PCR-RFLP.
